(19) United States
(12) Patent Application Publication
(10) Pub. No.: US 2025/0082188 A1
(43) Pub. Date: Mar. 13, 2025

(54) METHOD AND ARRANGEMENT FOR PRE-CLEANING AN ENDOSCOPE

(71) Applicant: Olympus Winter & Ibe GmbH, Hamburg (DE)

(72) Inventor: Benjamin Ottens, Hamburg (DE)

(73) Assignee: Olympus Winter & Ibe GmbH, Hamburg (DE)

(21) Appl. No.: 18/883,396

(22) Filed: Sep. 12, 2024

Related U.S. Application Data
(60) Provisional application No. 63/538,111, filed on Sep. 13, 2023.

(51) Int. Cl. A61B 1/12

(52) U.S. Cl. CPC A61B 1/12

(57) ABSTRACT

A method for pre-cleaning an endoscope. The method including: fluidly connecting channels for transporting fluids within an endoscope to each other at a distal end of the endoscope by arranging a container onto the distal end of the endoscope such that the channels are fluidly connected to each other at the distal end of the endoscope, the channels extending from a respective connection port to the distal end of the endoscope; connecting a suction pump to at least one of the connection ports; connecting at least one liquid source to the at least one of the connection ports; and flushing at least one of the channels with a liquid by activating the suction pump so that the liquid is drawn from the liquid source through the at least one channel via the distal end towards the suction pump.

### CROSS-REFERENCE TO RELATED APPLICATION

The present application is based upon and claims the benefit of priority from U.S. Provisional Application No. 63/538,111 filed on Sep. 13, 2023, the entire contents of which is incorporated herein by reference.

The present application is related to the following co-pending U.S, Applications, each filed on the same day, US. patent application Ser. No. 18/______ (corresponding to U.S. Provisional Patent Application 63/538,114); U.S. patent application Ser. No. 18/______ (corresponding to U.S. Provisional Patent Application 63/538,116); and U.S. patent application Ser. No. 18/______ (corresponding to U.S. Provisional Patent Application 63/538,119); the entire contents of each of which is incorporated herein by reference.

### BACKGROUND

### Field The present disclosure relates to a method for pre-cleaning an endoscope.

Further, the present disclosure relates to an arrangement for pre-cleaning an endoscope.

### Prior Art

An endoscope is an inspection instrument that can be used to examine, and optionally manipulate, the inside of organisms. Endoscopes can be used in human medical diagnostics for examinations or minimally invasive surgical procedures on humans or animals. Endoscopes typically comprise an image sensor, an optical lens, and a light source.

After use of an endoscope, the endoscope is typically pre-cleaned, manually or automated cleaned, disinfected, and possibly sterilized before being used again on a patient. If an endoscope and accessories used in the patient procedure are not immediately cleaned after each patient procedure, residual organic debris will begin to dry and solidify, hindering effective removal and reprocessing efficacy. Therefore, it is necessary to pre-clean the endoscope and the accessories at the bedside immediately after each patient procedure.

Typically, the pre-cleaning that is conducted at the bedside immediately after each patient procedure inter alia comprises the following steps: First, a biopsy/suction channel is pre-cleaned by arranging a distal end of the endoscope in water, and drawing the fluid through the endoscope. Then, air is drawn through the endoscope. Second, an air/water channel is pre-cleaned by arranging the distal end of the endoscope in water and flush the air/water channel with the fluid. Then, the air/water channel is flushed with air. Third, if available, an auxiliary water channel is pre-cleaned by flushing the auxiliary water channel with water. Finally, the accessories are detached from the endoscope and placed in a fluid, such as water or a detergent solution. Typically, after the pre-cleaning procedure the endoscope is detached from the equipment (such as for example video system center, light source, suction pump) used in the patient procedure and transported to a reprocessing area.

In U.S. Pat. No. 11,445,900 B2 it was suggested to pre-clean an endoscope by using a plurality of input members in the form of flexible tubes for use in delivering cleaning solution to multiple input ports of an endoscope, so that a cleaning solution is delivered through the flexible tubes to the endoscope.

However, known procedures for pre-cleaning are both relatively time-consuming and prone to errors. As the pre-cleaning typically consists of several subsequently conducted manually applied steps, the pre-cleaning is relatively time-consuming and if only one of these steps is not conducted exactly according to the pre-cleaning procedure, the endoscope might not be pre-cleaned sufficiently. Furthermore, because not all endoscope channels are connected to each other it is not possible to flush all channels with only one source. In typically conducted pre-cleaning procedures, the video system center is required to flush the air/water channel, a suction pump to aspire the suction and biopsy channel and a flushing pump or syringe to flush the auxiliary water channel. Therefore, the user is permanently involved in the procedure and needs to interact with several different user interfaces which requires that the user can operate all of these user interfaces. Additionally a lot of equipment/accessories is necessary to conduct the pre-cleaning procedure.

Furthermore, a problem is that after pre-cleaning, drying and solidification can occur in the channels that are no longer filled with fluid.

### SUMMARY

Therefore, an object is to provide an improved method and an improved arrangement for pre-cleaning an endoscope, for example, to provide a method for pre-cleaning an endoscope that is less prone to errors.

According to a first aspect, a method is provided for pre-cleaning an endoscope, for example, immediately or shortly after use of the endoscope. The method comprising: providing an endoscope that comprises channels for transporting fluids, connection ports, such as, for connection of fluid hoses, and a distal end of the endoscope, wherein each of the channels extends from one of the connection ports to the distal end of the endoscope; fluidly connecting the channels to each other at the distal end of the endoscope by arranging a container onto the distal end of the endoscope; connecting a suction device, such as, a suction pump, to one of the connection ports; connecting at least one liquid source to at least one of the connection ports; flushing at least one of the channels with a liquid by activating the suction device so that the liquid is drawn from the liquid source through the at least one channel via the distal end towards the suction device.

In the following, initially the method for pre-cleaning an endoscope and its functions and embodiments will be described.

The distal end of the endoscope may also be referred to as the distal tip of the endoscope. The distal end can be the end portion of the endoscope that is located at the very front of an insert section of the endoscope, such as, the end portion that is first inserted into the patient when the insert section of the endoscope is moved into a patient. Outlets of the channels can be arranged at the distal end. Further, a light guide can be arranged at the distal end. Still further, an objective lens can be arranged at the distal end. Still further yet, the distal end can be arranged at the end of an insertion section that has a flexible insertion tube.

The method can be conducted immediately or shortly after use of the endoscope in a patient procedure. The method for pre-cleaning the endoscope can be started within 60 minutes after being used in the patient procedure, such as within 30 minutes after being used in the patient procedure, or within 15 minutes after being used in the patient procedure. The pre-cleaning can be conducted at a patient bedside so that the method can be conducted immediately or shortly after use of the endoscope in a patient procedure without the need to move the endoscope to another location prior to conducting the pre-cleaning.

At least two of the channels can be flushed with a liquid by activating the suction device so that the liquid is drawn from the liquid source through at least one of the channels via the distal end and then from the distal end through at least one other channel of the channels towards the suction device.

The channels can each extend from one of the connection ports to the distal end of the endoscope. However, the endoscope can also have additional channels that do not extend from one of the connection ports to the distal end.

The connection ports can be ports to which fluid hoses or the like can be connected to.

The endoscope can for example be a colonoscope, or a gastroscopes, or an ultrasonic endoscope, or a duodenoscopes, or another type of endoscope.

The container can be arranged onto the distal end in a condition in which it is filled with liquid or in which it is not filled with liquid. If the container is not filled with liquid, a continuous flow of liquid can be drawn through the channels via the distal end.

Before flushing channels of the endoscope, the container may not be filled with liquid. In such a configuration, liquid can be drawn from a connection port through a channel to the container and from the container through a suction channel towards the suction device out of the endoscope, wherein no gaps of liquid that contain gas are present. Thus, when liquid is drawn out of a connection port, then the user can be sure that this liquid has been drawn completely through at least two channels. However, alternatively, it is also possible that the container is already filled with liquid before flushing the channels. In such a configuration, the distal end of the endoscope can be in contact with liquid for a longer period of time and soaked for a longer period of time, which can be beneficial to achieve a more thorough pre-cleaning of the distal end, if needed.

Even though not all channels of the endoscope are connected to each other inside of the endoscope, it is possible to flush all of these channels with only one source by connecting the channels to each other via the container that is arranged onto the distal end of the endoscope. Therefore, the pre-cleaning procedure can be conducted faster, easier and in a way that is less prone to errors.

The channels can be kept humid and/or filled with liquid after having conducted the pre-cleaning procedure. Thus, drying and solidification in the channels after pre-cleaning can be prevented.

During conducting the pre-cleaning of the endoscope it is not necessary to interact with several user interfaces and/or devices in order to conduct the pre-cleaning procedure.

Compared to conventional pre-cleaning methods, with the described method, much less equipment and/or accessories is necessary to conduct the pre-cleaning. For example, no video center or flushing pump is necessary to conduct the pre-cleaning.

In an embodiment, connecting at least one liquid source to at least one of the connection ports can comprise connecting at least one vessel that holds liquid, such as at least three, or four, vessels that hold liquid, to at least one of the connection ports of the connection ports.

By connecting at least one vessel that holds liquid to at least one of the connection ports and applying an underpressure, the liquid can be drawn from the vessel through the channel that extends from the connection port to which the vessel is connected to the distal end of the endoscope and because the container is arranged at the distal end, the liquid can further be drawn from the distal end through another channel, such as towards a suction device. This way it can be achieved that the liquid is drawn through the entire extension of both of these channels starting from the vessel, through the first channel via the distal end and then through the other channel, so that both of these channels are reliably flushed with the liquid along their whole extension. Further channels can be reliably flushed in the same way. Therefore, the vessels can be arranged onto several connection ports so that all of the channels that extend from these connection ports to the distal end can reliably be flushed with liquid along their whole extension.

In an embodiment, the method can comprise connecting closure elements to each of the connection ports to which no liquid source and no suction device is connected.

Each of the connection ports can be connected either to a vessel or to a closure element or to a suction device. For example, then each of the connection ports can be connected to a vessel or to a closure element or, for example indirectly via a hose or the like, to a suction device.

Closure elements can be elements that are configured to close a connection port, such as fluid-tight/air-tight, when being applied onto the connection port. Closure elements can be connected to such connection ports that are connected to a channel that does not need to be flushed in the pre-cleaning procedure.

In a further embodiment, the method can comprise flushing at least two of the channels, such as all of the channels, with liquid by activating the suction device so that liquid is drawn through the channels via the distal end towards the suction device.

By activating the suction device, an underpressure can be obtained with which the liquid can be drawn through the cannels towards the suction device. Only one of the channels can be connected to the suction device.

In a further embodiment, the channels can comprise an air channel that extends from an air channel connection port to the distal end of the endoscope, a suction channel that extends from a suction channel connection port to the distal end of the endoscope, a water channel that extends from a water channel connection port to the distal end of the endoscope, and/or an auxiliary water channel that extends from an auxiliary water channel connection port to the distal end of the endoscope.

The air channel can be configured to provide air to the distal end of the endoscope. The suction channel can be configured to draw liquids and/or objects, such as for example tissue samples, from the distal end of the endoscope through the endoscope. The water channel can be configured to provide water at the distal end of the endoscope. The auxiliary water channel can be configured to provide water at the distal end of the endoscope.

The channels can be arranged parallel to each other over at least a part of their extension from one of the connection ports to the distal end of the endoscope.

In a further embodiment, the container can be arranged onto the distal end of the endoscope such that the distal end, including the outer surface of the distal end, is arranged entirely inside the container.

The outer surface, such as that formed as an outer circumferential surface, of the distal end can be arranged inside of the container when the container is filled with fluid.

In a further embodiment, the container can be filled with liquid and the whole distal end, including the outer surface of the distal end, can be in contact with the liquid.

The pre-cleaning of the distal end can be challenging, especially if the distal end has a complex geometry, which is for example the case for duodenoscopes. By arranging the whole distal end in the container and filling the container with fluid, the distal end and its outer surface can reliably be pre-cleaned because the liquid then is in contact with all surfaces, including the outer surface, of the distal end.

In a further embodiment, the container can be arranged on the distal end of the endoscope such that the distal end of the endoscope can be sealed liquid-tight, and can be gas-tight, from the environment that surrounds the container.

By sealing the inside of the container against the environment, no external fluids can enter the channels and no liquid can flow out from the container in the environment that surrounds the container.

The inner volume of the container does not have to be changeable. This can be achieved, for example, by using a relatively stiff material for the wall of the container that defines an inner chamber of the container. This way, when an underpressure is applied to the inner chamber of the container, the inner volume of the container remains substantially unchanged so that the fluid connection of the channels at the end of the endoscope remains even when an underpressure is applied to the channel system and thus to the inner volume of the container.

In a further embodiment, each of the connection ports can be connected to a vessel, except for the connection port that is connected to the suction device.

By arranging the vessels to all connection ports except for the connection port that is connected to the suction device, all channels can reliably be flushed and thus pre-cleaned, for example, if all channels are to be flushed.

In a further embodiment, the suction device can be fluidly connected to a suction container that is configured to receive liquid drawn out of the endoscope by the suction device.

By connecting such a suction container to the suction device, the liquid drawn through the channels and out of the endoscope via the suction device can be safely collected.

In a further embodiment, the liquid that is drawn through at least one of the channels can be water or a disinfectant solution or a detergent solution.

For example, a disinfectant solution and/or detergent solution can be used in order to achieve a better removal of organic debris when compared to water.

In a further embodiment, the endoscope can be immersed in a liquid so that the connection ports are in contact with the liquid, for example, the connection port that is connected to the suction device can be connected to the suction device via a hose or the like.

In such an alternative solution compared to a solution in which vessels are arranged onto connection ports, the endoscope can fully or partly be immersed in a liquid so that the connection ports are in contact with the liquid in which the endoscope is immersed in.

The connection port that is connected to the suction device can be connected to the suction device via a hose or the like. This connection port can also be immersed in the liquid, but without providing a fluid connection between the channel that is connected to the connection port and the liquid.

According to a further aspect, an arrangement for pre-cleaning an endoscope, such as immediately or shortly after use of the endoscope, is provided. The arrangement comprising: an endoscope that comprises channels for transporting fluids, connection ports, such as for connection of fluid hoses, and a distal end of the endoscope, wherein each of the channels extends from one of the connection ports to the distal end of the endoscope; a container that is arranged onto the distal end of the endoscope such that the channels are fluidly connected to each other at the distal end of the endoscope; at least one liquid source that is connected to at least one of the connection ports; and for example a suction device, such as a suction pump, that is connected to one of the connection ports.

The advantages explained above with respect to the method for pre-cleaning are also advantages for such an arrangement for pre-cleaning. For example, the advantages can be achieved by arranging the container onto the distal end such.

In a further embodiment, the suction device can be configured to flush at least one of the channels with a liquid by activating the suction device so that the liquid is drawn from the liquid source through the at least one channel via the distal end towards the suction device.

In a further embodiment, the endoscope can comprise: an air channel that extends from an air channel connection port to the distal end of the endoscope, a suction channel that extends from a suction channel connection port to the distal end of the endoscope, a water channel that extends from a water channel connection port to the distal end of the endoscope, and/or an auxiliary water channel that extends from an auxiliary water channel connection port to the distal end of the endoscope, and can further comprise at least one closure element that is connected to each of the connection ports to which no liquid source and no suction device is connected.

As to the advantages, embodiments and details of the individual different aspects and their embodiments, reference is also made to the corresponding embodiments and details described with reference to the respective other aspects.

Further embodiments can result from the combination of individual, several or all of the features described herein.

### BRIEF DESCRIPTION OF THE DRAWINGS

The embodiments are described with reference to the attached drawings, in which:

FIG. 1 illustrates a schematic depiction of an exemplary embodiment of an arrangement for pre-cleaning an endoscope;

FIG. 2 illustrates a schematic flow diagram of an exemplary embodiment of a method for pre-cleaning an endoscope; and

FIG. 3 illustrates a schematic flow diagram of an exemplary embodiment of a method for pre-cleaning an endoscope.

### DETAILED DESCRIPTION

In the Figures, elements with the same or comparable functions are indicated with the same reference numerals.

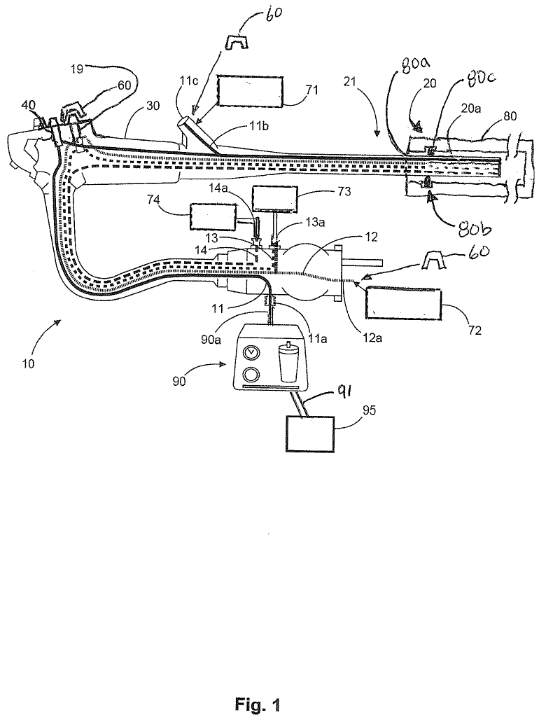

FIG. 1 shows a schematic depiction of an exemplary embodiment of an arrangement for pre-cleaning an endoscope 10. The endoscope 10 can be pre-cleaned immediately or shortly after use of the endoscope 10. The endoscope 10 comprises an endoscope body 30 and channels 11, 12, 13, 14 for transporting fluids, namely a suction channel 11, an air channel 12, a water channel 13, and an auxiliary water channel 14.

The suction channel 11 is configured to draw liquids and/or objects, such as for example a tissue sample, from the distal end 20 of the endoscope 10 through the endoscope 10. The suction channel 11 extends from a suction channel connection port 11a to the distal end 20 of the endoscope 10. The distal end 20 is the end portion of the endoscope 10 that is located at the very front of an insert section 21 of the endoscope 20.

The air channel 12 is configured to provide air to the distal end 20 of the endoscope 10. The air channel 12 extends from an air channel connection port 12a to the distal end 20 of the endoscope 10.

The water channel 13 is configured to provide water at the distal end 20 of the endoscope 10. The water channel 13 extends from a water channel connection port 13a to the distal end 20 of the endoscope 10.

The auxiliary water channel 14 is configured to provide water at the distal end 20 of the endoscope 10. The auxiliary water channel 14 extends from an auxiliary water channel connection port 14a to the distal end 20 of the endoscope 10.

The connection ports 11a, 11c, 12a, 13a, 14a are configured for connection of fluid hoses and/or vessels and/or closure elements. Each of the channels 11, 12, 13, 14 extends from one of the connection ports 11a, 12a, 13a, 14a to the distal end 20 of the endoscope 10.

The channels 11, 12, 13, 14 are fluidly connected to each other at the distal end 20 of the endoscope 10 by arranging a container 80 onto the distal end 20 of the endoscope 10. The container 80 is arranged onto the distal end 20 of the endoscope 10 such that the distal end 20, including the outer circumferential surface 20a of the distal end 20, is arranged entirely inside the container 80. For example, the container 80 can have a hole 80a having a similar cross-sectional shape as the distal end 20 of the endoscope and the hole 80a can be provided with a seal 80b. For example, the distal end of the endoscope can have a round cross-section with the hole 80a having an O-ring seal 80c within the hole. The outer shape of the container 80 can have any shape, such as a cylinder or cube. If the container 80 gets filled with liquid, the whole distal end 20, including the outer surface 20a of the distal end 20, is in contact with the liquid so that a reliable pre-cleaning of the whole distal end 20 can be achieved. The container 80 is arranged on the distal end 20 of the endoscope 10 such that the distal end 20 of the endoscope 10 is sealed liquid-tight and gas-tight from the environment that surrounds the container 80.

The endoscope 10 furthermore comprises a biopsy branch 11b that extends from a biopsy branch connection port 11c to the suction channel 11.

The air channel 12 and the water channel 13 can be fluidly connected and/or separated from each other via an air/water cylinder 19. A vent can be assembled to the air/water cylinder 19. The air/water cylinder 19 is sealed with a closure element 60 that is configured to close the air/water cylinder 19 fluid-tight. Furthermore, a suction cylinder 40 is provided to which the suction channel 11 is connected to. A vent can be assembled to the suction cylinder 40.

A vessel 71 is arranged onto the biopsy branch connection port 11c. The vessel may have any shape. The vessel may have a similar sealing configuration as discussed with regard to the container 80. Alternatively, the vessel can have an extending hose with a sealing connector nut for connection to the biopsy branch connection portion 11c. Such connector nuts are well known in the art. If the biopsy branch 11b does not need to be flushed during the pre-cleaning procedure, a closure element can be arranged onto the biopsy branch connection port 11c instead of the vessel 71. The closure element 60 can have a similar connection configuration as the connector nut described above, except having a closed end to close off the biopsy branch connection portion 11c.

A vessel 72 is arranged onto the connection port 12a of the air channel 12. If the air channel 12 does not need to be flushed during the pre-cleaning procedure, a closure element 60 can be arranged onto the connection port 12a instead of the vessel 72.

A vessel 73 is arranged onto the connection port 13a of the water channel 13. If the water channel 13 does not need to be flushed during the pre-cleaning procedure, a closure element 60 configured to mate with and close the connection port 13a can be arranged onto the connection port 13a instead of the vessel 73.

Furthermore, a vessel 74 is arranged onto the connection port 14a of the auxiliary water channel 14. If auxiliary water channel 14 does not need to be flushed during the pre-cleaning procedure, a closure element 60 configured to mate with and close the connection port 14a can be arranged onto the connection port 14a instead of the vessel 74.

The vessels 71, 72, 73, 74 are filled with liquid, such as with a disinfectant solution and/or detergent solution. Each of the vessels 72, 73 and 74 can be similarly configured as discussed above with regard to vessel 71. Additionally, the closure element for each of vessels 72, 73 and 74 can be similarly configured as discussed above with regard to the closure element for vessel 71.

A suction device 90, that can be a suction pump, is connected via a fluid hose 90a to the connection port 11a of the suction channel 11. When the suction device 90 is activated, an underpressure is applied to the system so that liquid is drawn towards the suction device 90 as described in detail below. As used herein “underpressure” means a negative pressure is applied by the suction device 90.

Liquid is drawn from the vessel 71 through the biopsy branch 11b and from there further through the suction channel 11, out of the connection port 11a, via the fluid hose 90a to the suction device 90 and from the suction device 90 to the suction jar 95 via fluid hose 91.

Liquid is drawn from the vessel 72 through the air channel 12 to the distal end 20. Due to the fluid connection at the distal end 20 that is provided by the container 80 arranged at the distal end 20, the liquid is drawn from there through the whole extension of the suction channel 11, out of the connection port 11a, via the fluid hose 90a to the suction device 90 and from the suction device 90 to the suction jar 95.

Liquid is drawn from the vessel 73 through the water channel 13 to the distal end 20. Due to the fluid connection at the distal end 20 that is provided by the container 80 arranged at the distal end 20, the liquid is drawn from there through the whole extension of the suction channel 11, out of the connection port 11a, via the fluid hose 90a to the suction device 90 and from the suction device 90 to the suction jar 95.

Liquid is drawn from the vessel 74 through the auxiliary water channel 14 to the distal end 20. Due to the fluid connection at the distal end 20 that is provided by the container 80 arranged at the distal end 20, the liquid is drawn from there through the whole extension of the suction channel 11, out of the connection port 11a, via the fluid hose 90a to the suction device 90 and from the suction device 90 to the suction jar 95.

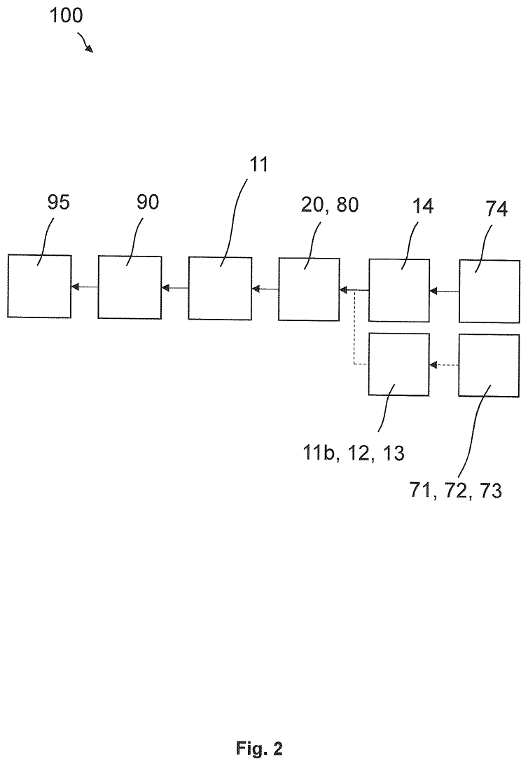

FIG. 2 shows a schematic flow diagram of an exemplary embodiment of a method 100 for pre-cleaning an endoscope 10 that is configured as shown in FIG. 1. The method 100 for pre-cleaning the endoscope 10 comprises fluidly connecting the channels 11, 11b, 12, 13, 14 of the endoscope to each other at the distal end of the endoscope by arranging a container onto the distal end 20 of the endoscope and connecting a suction device 90 to a channel 11, wherein the suction device 90 is further connected to a suction container, formed as a suction jar 95. Vessels 74, 71, 72, 73 that contain liquid are arranged onto the connection ports of the channels 11b, 12, 13, 14. By activating the suction device 90, liquid is drawn in the direction indicated by the arrows, namely from the vessels 74, 71, 72, 73 through the channels 11b, 12, 13, 14 (to which the vessels 74, 71, 72, 73 are connected to) towards the distal end 20 and from the distal end 20 via the container 80 into the suction channel 11 and through the suction channel 11 and through the suction channel to the suction pump 90 to be then collected in the suction jar 95.

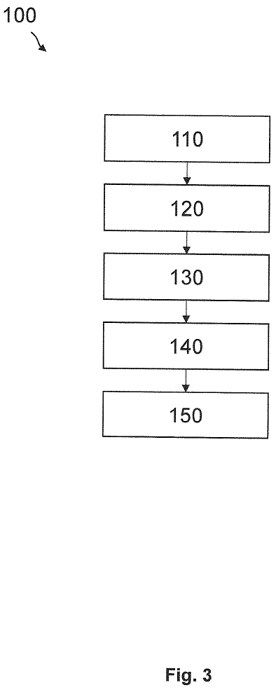

FIG. 3 shows a schematic flow diagram of an exemplary embodiment of a method 100 for pre-cleaning an endoscope as shown and described in connection with FIG. 1. The method 100 is conducted immediately or shortly after use of the endoscope in a patient procedure and comprises the following steps:

In a step 110, providing an endoscope that comprises channels for transporting fluids, connection ports, such as for connection of fluid hoses, and a distal end of the endoscope, wherein each of the channels extends from one of the connection ports to the distal end of the endoscope.

In a step 120, fluidly connecting the channels to each other at the distal end of the endoscope by arranging a container onto the distal end of the endoscope.

In a step 130, connecting a suction device, such as a suction pump, to one of the connection ports.

In a step 140, connecting at least one liquid source to at least one of the connection ports.

In a step 150, flushing at least one, such as at least two, of the channels with a liquid by activating the suction device so that the liquid is drawn from the liquid source through the at least one channel, such as at least two channels, via the distal end towards the suction device.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A method for pre-cleaning an endoscope, the method comprising:
fluidly connecting channels for transporting fluids within an endoscope to each other at a distal end of the endoscope by arranging a container onto the distal end of the endoscope such that the channels are fluidly connected to each other at the distal end of the endoscope, the channels extending from a respective connection port to the distal end of the endoscope;
connecting a suction pump to at least one of the connection ports;
connecting at least one liquid source to the at least one of the connection ports; and
flushing at least one of the channels with a liquid by activating the suction pump so that the liquid is drawn from the liquid source through the at least one channel via the distal end towards the suction pump.

2. The method according to claim 1, wherein the connecting at least one liquid source to the at least one of the connection ports comprises connecting at least one vessel that holds liquid to the at least one of the connection ports.

3. The method according to claim 1, wherein the connecting the at least one liquid source to the at least one of the connection ports comprises connecting two or more vessels that each hold liquid to two or more respective connection ports.

4. The method according to claim 1, further comprising connecting closure elements to each of the connection ports to which no liquid source and no suction pump is connected.

5. The method according to claim 1, wherein the flushing comprises flushing two or more of the channels with the liquid by activating the suction pump so that liquid is drawn through the two or more channels via the distal end towards the suction pump.

6. The method according to claim 1, wherein the channels comprise two or more of:
an air channel that extends from an air channel connection port to the distal end of the endoscope,
a suction channel that extends from a suction channel connection port to the distal end of the endoscope,
a water channel that extends from a water channel connection port to the distal end of the endoscope, and
an auxiliary water channel that extends from an auxiliary water channel connection port to the distal end of the endoscope.

7. The method according to claim 1, wherein the arranging of the container onto the distal end of the endoscope comprises arranging the container such that the distal end, including the outer surface of the distal end, is arranged entirely inside the container.

8. The method according to claim 7, wherein the container is filled with liquid and the whole distal end, including the outer surface of the distal end, is in contact with the liquid.

9. The method according to claim 1, wherein the arranging of the container on the distal end of the endoscope comprises arranging the container such that the distal end of the endoscope is one of sealed liquid-tight or sealed gas-tight, from an environment that surrounds the container.

10. The method according to claim 1, wherein each of the connection ports is connected to a vessel, except for the at least one of the connection ports that is connected to the suction pump.

11. The method according to claim 1, wherein the suction pump is fluidly connected to a suction container that is configured to receive liquid drawn out of the endoscope by the suction pump.

12. The method according to claim 1, wherein the liquid that is drawn through at least one of the channels is one of water, a disinfectant solution, or a detergent solution.

13. The method according to claim 1, wherein the at least one liquid source comprises immersing the endoscope in a liquid so that the connection ports are in contact with the liquid.

14. The method according to claim 13, wherein the connection port connected to the suction pump is connected to the suction pump via a hose.

15. An arrangement for pre-cleaning an endoscope comprising:
a container arranged onto a distal end of an endoscope such that channels for transporting fluids within the endoscope are fluidly connected to each other at a distal end of the endoscope, the channels extending from a respective connection port to the distal end of the endoscope;
at least one liquid source connected to at least one of the connection ports; and
a suction pump connected to one of the connection ports.

16. The arrangement according to claim 15, wherein the suction pump is configured to flush at least one of the channels with a liquid by activating the suction pump so that the liquid is drawn from the liquid source through the at least one channel via the distal end towards the suction pump.

17. The arrangement according to claim 15, further comprising the endoscope.

18. The arrangement according to claim 17, further comprising closure elements connected to each of the connection ports to which no liquid source and no suction pump is connected.
